(19) United States
(12) Patent Application Publication
(10) Pub. No.: US 2018/0243387 A1
(43) Pub. Date: Aug. 30, 2018

(54) TECHNOLOGY FOR CONTROLLING IMMUNE CELLS

(71) Applicant: RIKEN, Wako-shi (JP)

(72) Inventors: Takashi SAITO, Wako-shi (JP); Takayuki IMANISHI, Wako-shi (JP)

(21) Appl. No.: 15/897,628

(22) Filed: Feb. 15, 2018

(30) Foreign Application Priority Data
Feb. 17, 2017 (JP) 2017-028244

(51) Int. Cl. A61K 39/00; A61K 39/39

(52) U.S. Cl. CPC A61K 39/0008; A61K 2039/57; A61K 39/39

(57) ABSTRACT

The invention provides compositions or pharmaceutical compositions for use in inducing acquired immune lymphocytes to produce type I interferons, inhibiting proliferation of the lymphocytes, and treating diseases or conditions associated with enhanced acquired immunity. Pharmaceutical compositions for use in treating diseases or conditions associated with enhanced acquired immunity, containing a STING ligand; compositions for use in inducing T cells to produce type I interferons, containing a STING ligand; and proliferation inhibitors of acquired immune lymphocytes containing a STING ligand are provided.

### BACKGROUND OF THE INVENTION

### Field of the Invention

The present invention relates to a technology for controlling activated immune cells and, in particular, to compositions or pharmaceutical compositions for use in inducing acquired immune lymphocytes to produce type I interferons, inhibiting proliferation of the lymphocytes, and treating diseases or conditions associated with enhanced acquired immunity.

### Description of the Related Art

Studies on various autoimmune diseases and development of therapies thereof have been conducted. In autoimmune diseases, acquired immunity is activated to a self-antigen and can attack self. Therefore, autoimmune diseases have been treated with immunosuppressive agents.

By virtue of the progress in studies on innate immunity, it has been found that invasion of a pathogen (for example, bacterium, virus, fungus) into the living body results in recognition of the pathogen by pattern recognition and the removal of the pathogen by innate immunity. Toll-like receptors (TLRs) have been discovered as a factor involved in the pattern recognition and the mechanism of the recognition of pathogens on the cell surface has been revealed. Sensors for components of pathogens have been discovered also in cells. For example, RIG-I-like receptors (RLRs) have been found as an intracellular RNA sensor in innate immune cells and STING has been found as an intracellular nucleic acid sensor in innate immune cells.

STING is known to be involved in the intracellular nucleic acid recognition in innate immune cells and to recognize a cyclic dinucleotide that is produced by processing of nucleic acid from pathogens (Science, Vol. 339 (6121), 786-791, 2013).

### SUMMARY OF THE INVENTION

The present invention provides a technology for controlling activated immune cells and, in particular, compositions or pharmaceutical compositions for use in inducing acquired immune lymphocytes to produce type I interferons, inhibiting proliferation of the lymphocytes, and treating diseases or conditions associated with enhanced acquired immunity. In particular, the present invention provides a technology for controlling activated immune cells.

The present inventors have discovered that STING ligands that activate the innate immune system have the effect of inhibiting the proliferation of acquired immune lymphocytes and the effect of inducing acquired immune lymphocytes to produce type I interferons and these effects are prominent in the presence of the proliferation stimulation by TCR signals and thereby completed the present invention.

Thus, according to the present invention, the following invention is provided.

(1) A pharmaceutical composition for use in treating a disease or condition associated with enhanced acquired immunity, comprising a STING ligand.
(2) The pharmaceutical composition according to (1) above, wherein the disease or condition associated with enhanced acquired immunity is an autoimmune disease or a graft versus host disease (GVHD).
(3) The pharmaceutical composition according to (1) or (2) above, for prophylactically treating the onset of the disease or condition associated with enhanced acquired immunity.
(4) The pharmaceutical composition according to any of (1) to (3) above, wherein the STING ligand is cyclic GMP-AMP.
(5) A composition for use in inducing a T cell to produce a type I interferon, comprising a STING ligand.
(6) A proliferation inhibitor of acquired immune lymphocytes, comprising a STING ligand.

### BRIEF DESCRIPTION OF THE DRAWINGS

FIG. 1 illustrates the effect of treating naïve CD4 cells with cGAMP under CD3 and CD28 stimulation on cell proliferation and the involvement of STING therein.

FIG. 2 illustrates the result of measuring the cell survival rate after adding cGAMP and etoposide, a cell death inducer, to naïve CD4 cells.

FIG. 3 illustrates change in gene expression of cell cycle regulators in naïve CD4 cells after the cGAMP treatment.

FIG. 4 illustrates the effect of cGAMP on change in intracellular signals after CD3 and CD28 stimulation.

FIG. 5 illustrates change in expression of lipid metabolism-associated genes in naïve CD4 cells after the cGAMP treatment.

FIG. 6A illustrates the effect of cGAMP on the production of the type I interferon in naïve CD4 cells after CD3 and CD28 stimulation and the involvement of Tbk1 therein.

FIG. 6B illustrates the effect of cGAMP on cell proliferation of naïve CD4 cells after CD3 and CD28 stimulation and the involvement of Tbk1 therein.

FIG. 7A illustrates the effect of cGAMP on cell proliferation in naïve CD4 cells after CD3 and CD28 stimulation and the involvement of IRF3 and IRF7 therein.

FIG. 7B illustrates the effect of cGAMP on the production of the type I interferon in naïve CD4 cells after CD3 and CD28 stimulation and the involvement of IRF3 and IRF7 therein.

FIG. 8 illustrates the effect of cGAMP on the expression of lipid metabolism-associated genes in naïve CD4 cells after CD3 and CD28 stimulation and the involvement of IRF3 and IRF7 therein.

### DETAILED DESCRIPTION OF THE PREFERRED EMBODIMENTS

As used herein, the “subject” means a mammal and may be, in particular, a human.

As used herein, the “treatment” is used in a meaning including therapy (therapeutic treatment) and prophylaxis (prophylactic treatment). As used herein, the “therapy” means therapy, cure, prevention, or improvement of remission of a disease or disorder or reduction of progression speed of a disease or disorder. As used herein, the “prophylaxis” means reducing the possibility of the onset of a disease or pathological condition or delaying the onset of a disease or pathological condition.

As used herein, the “disease” means a symptom to which a therapy is useful. Herein, diseases include chronic and acute diseases.

As used herein, the “disease or condition associated with enhanced acquired immunity” is used in a meaning including a disease or symptom caused by abnormality in the acquired immune system or incompatibility in the immune system and in particular a disease or symptom whose condition is aggravated by enhanced acquired immunity. The “disease or condition associated with enhanced acquired immunity” is used in a meaning including a disease or the symptom associated with enhanced acquired immunity. Examples of the disease or condition associated with enhanced acquired immunity include autoimmune diseases, immunorejection after tissue transplantation, immunorejection after hematopoietic stem cell transplantation or blood transfusion (for example, graft versus host disease (GVHD)), and immunorejection upon entry of a foreign material.

As used herein, the “autoimmune disease” means a disease caused by an enhanced immune response to self. Examples of the autoimmune diseases include rheumatoid arthritis, insulin-dependent diabetes mellitus, multiple sclerosis, lupus, psoriasis, inflammatory bowel disease, ulcerative colitis, myasthenica gravis, polymyositis, dermatomyositis, autoimmune cytopenia, vasculitic syndrome, systemic lupus erythematosus, and the like.

As used herein, the “therapeutically effective amount” means an amount of an agent effective for the treatment (prophylaxis or therapy) of a disease or condition. The therapeutically effective amount of the agent can reduce the aggravation speed of a symptom of the disease or condition, stopping the aggravation of the symptom, ameliorating the symptom, curing the symptom, or inhibiting the onset or development of the symptom.

As used herein, “STING” is a membrane protein on the endoplasmic reticulum and known as a DNA sensor in the cytoplasm. STING recognizes nucleic acids or nucleic acid derivatives from pathogens and activates the downstream signaling (nucleic acids or nucleic acid derivatives from pathogens that bind and activate STING may be referred to as “STING ligands”). Moreover, STING recognizes and is activated by STING ligands that bind to STING such as nucleic acids or nucleic acid derivatives such as cyclic [G(2′,5′)pA(3′,5′)p] (also referred to as 2′3′-cGAMP or simply cGAMP), cyclic diguanosine monophosphate (also referred to as c-di-GMP), cyclic diadenosine monophosphate (also referred to as c-di-AMP), and cyclic [G(3′,5′)pA(3′,5′)p] (also referred to as 3′3′-cGAMP) or low molecular weight agonists such as 5,6-dimethylxanthenone-4-acetic acid (DMX AA) and xanthenone derivatives. A STING ligand may be a natural nucleic acid or nucleic acid derivative or an unnatural nucleic acid or nucleic acid derivative. The activation of STING results in the activation of TBK1, the activation of IRF3, and the activation of IRF7 and the transduction of the signals into the nucleus to change the gene expression in the cell.

As used herein, “acquired immunity” is immunity attained by humoral immunity by antibodies and cell-mediated immunity by cells (T cells and B cells) such as lymphocytes. As used herein, acquired immune lymphocytes mean B cells and T cells having antigen specificity. As used herein, the “antigen specificity” means responding more strongly to a particular antigen than to other antigens. The antigen specificity is attained by T-cell receptors (TCRs) in T cells and by B-cell receptors (BCRs) in B cells. Acquired immunity is antigen-specific immunity and a type of immunity that adapts to foreign materials and acquires the ability to remove the foreign materials in the meaning that it is maintained by memory cells, although its immune responses take time, and when encountered to the same foreign materials again it can immediately remove the foreign materials.

As used herein, the “innate immunity” is immunity that occurs as the first biological response to foreign materials and involves phagocytosis by macrophages, granulocytes, or NK cells. Examples of diseases whose main cause is abnormality of the innate immunity include autoinflammatory diseases. The autoinflammatory disease is defined by the 3 main characteristics: inflammation from unknown causes, the absence of high-titer autoantibodies and autoreactive T cells, and congenital abnormality of innate immunity, and examples thereof include diseases such as Muckle-Wells syndrome, familial cold autoinflammatory syndrome (FACS), and cryopyrin-associated periodic syndrome (CAPS). Such autoimmune diseases whose main cause is considered to be abnormality of the innate immunity may be excluded from the diseases to be treated with a therapy according to the present invention.

As used herein, the “type I interferons” means antiviral cytokines such as interferon α (INF-α) and interferon β (INF-β) among interferons. Interferon γ (INF-γ) is classified as a type II interferon and distinguished from the type I interferons. The type I interferons are known for the antivirus effect and remove viruses from the body by inhibition of the viral replication, the protection of noninfected cells, and the promotion of removal of infected cells by NK cells.

According to the present invention, STING ligands can inhibit the proliferation of acquired immune lymphocytes. Therefore, according to the present invention, proliferation inhibitors of acquired immune lymphocytes, comprising a STING ligand are provided. The proliferation inhibitors according to the present invention have strong inhibiting effects and can exhibit the effect of inhibiting cellular proliferation even when cells have received proliferation stimulation (in particular, T-cell receptor stimulation or T-cell stimulation or B-cell receptor stimulation or B-cell stimulation). Therefore, the proliferation inhibitors of acquired immune lymphocytes according to the present invention may be used in the presence of cell proliferation stimulation.

Examples of the proliferation stimulation for T cells include T-cell receptor (TCR) stimulation, T-cell stimulation by stimulation of a T-cell co-stimulatory receptor by agonists such as anti-CD3 antibodies and/or anti-CD28 antibodies, T-cell stimulation by ionomycin, and T-cell stimulation by phytohemagglutinin (PHA) and/or a phorbol ester, for example, phorbol myristate acetate (PMA), 12-O-tetradecanoylphorbol 13-acetate (TPA), and the like. The TCR stimulation may be provided with a major histocompatibility complex (MHC or HLA) that presents an antigen. Examples of the proliferation stimulation for B cells include B-cell receptor (BCR) stimulation, B-cell stimulation by stimulation of a B-cell co-stimulatory receptor such as CD21, CD19, and CD81, CD40 stimulation, BAFF receptor stimulation, and the like. BCR may be stimulated by an antigen or an anti-immunoglobulin antibody. CD40 may be stimulated by a CD40 ligand or an anti-CD40 antibody. The BAFF receptor may be stimulated by BAFF.

According to the present invention, STING ligands act on mTORC1 in the T-cell receptor signals, inhibit lipid metabolism and/or expression of cell cycle regulators, and thereby inhibit cellular proliferation. Therefore, according to the present invention, compositions or pharmaceutical compositions for use in inhibiting the expression of each of lipid synthesis-related factors (for example, Scd1, Acsl6, Elovl6, Lss, Sqle, Hmgcs1, Sc4 mol, and Cyp51) and/or cell cycle regulators (for example, cyclin A2, cyclin B1, cyclin D3, Cdk1, and Cdk4), which are activated in the presence of T-cell receptor signals, comprising a STING ligand are provided. Moreover, according to the present invention, compositions or pharmaceutical compositions for use in activating the expression of each of lipid metabolism and/or a cell cycle regulator (for example, p21), which are inhibited in the presence of T-cell receptor signals, comprising a STING ligand are provided. Here, the T-cell receptor signals include, in addition to TCR signals that are activated by stimulating TCR, downstream signals of TCR that are activated by stimulating cofactors of TCR, and downstream signals of TCR that are activated by stimulating T cells. According to the present invention, cell proliferation inhibitors of T cells, comprising a STING ligand are provided.

Moreover, the T-cell receptor signals correspond with the B-cell receptor signals in B cells. It is known that activation signal pathways of T cells and B cells are similar. Activation of either of these signals starts with the phosphorylation of an ITAM adapter (CD3, CD79) that associates with TCR (T cell receptor) or BCR (B cell receptor) by Src kinase. By the phosphorylation of the ITAM adapter, a kinase (ZAP70, Syk) that binds to the phosphorylated adapter is activated to activate further downstream adapters (LAT/SLP76, BLNK), and activate the NFAT/Ca²⁺ pathway, the NF-κB pathway, the Ras-MAPK pathway, and the like. Moreover, it is reported that mTORC1 plays an important role in the activation and functional differentiation of B cells, like T cells. Therefore, according to the present invention, compositions or pharmaceutical compositions for use in inhibiting the expression of each of lipid synthesis-related factors (for example, Scd1, Acsl6, Elovl6, Lss, Sqle, Hmgcs1, Sc4 mol, and Cyp51) and/or cell cycle regulators (for example, cyclin A2, cyclin B1, cyclin D3, Cdk1, and Cdk4), which are activated in the presence of B-cell receptor signals, comprising a STING ligand are provided. Moreover, according to the present invention, compositions or pharmaceutical compositions for use in activating the expression of each of lipid metabolism and/or cell cycle regulators (for example, p21), which are inhibited in the presence of B-cell receptor signals, comprising a STING ligand are provided. Here, the B-cell receptor signals include, in addition to BCR signals that are activated by stimulating BCR, downstream signals of BCR that are activated by stimulating cofactors of BCR and downstream signals of BCR that are activated by stimulating B cells. According to the present invention, cell proliferation inhibitors of B cells, comprising a STING ligand are provided.

Moreover, according to the present invention, STING ligands promote the production of type I interferons in acquired immune lymphocytes. Therefore, according to the present invention, compositions or pharmaceutical compositions for use in promoting production of type I interferons in acquired immune lymphocytes, comprising a STING ligand are provided. According to the present invention, cGAMP may be used with CD3 and CD28 stimulation.

According to the present invention, STING ligands can be used to treat diseases or conditions associated with enhanced acquired immunity through the proliferation inhibition of acquired immune lymphocytes. Therefore, according to the present invention, pharmaceutical compositions for use in treating diseases or conditions associated with enhanced acquired immunity, comprising a STING ligand are provided.

According to the present invention, STING ligands exert the effect of inhibiting the proliferation of acquired immune lymphocytes even in the presence of proliferation stimulation. Therefore, according to the present invention, the pharmaceutical compositions for use in treating diseases or conditions associated with enhanced acquired immunity, comprising a STING ligand may be used before, during, or after the cell proliferation stimulation. In this sense, the pharmaceutical compositions according to the present invention may be administered before the onset of the diseases or conditions associated with enhanced acquired immunity (for example, prophylactically). The pharmaceutical compositions according to the present invention may be administered during the development of the diseases or conditions associated with enhanced acquired immunity (for example, prophylactically or therapeutically). Furthermore, the pharmaceutical compositions according to the present invention may be used to therapeutically treat diseases or conditions associated with enhanced acquired immunity.

Examples of diseases or conditions suitable for the prophylactic administration of the pharmaceutical compositions according to the present invention include immunorejection after tissue transplantation, immunorejection after hematopoietic stem cell transplantation (for example, graft versus host disease (GVHD)), and immunorejection upon entry of a foreign material. Since these diseases or conditions are phenomena that occur after human intervention, the occurrence of the diseases or conditions can be predicted and these diseases or conditions are considered to be suitable for prophylactic administration of a STING ligand before, during, or after artificial intervention. In addition, the pharmaceutical compositions of the present invention may also be useful for immune diseases (including autoimmune diseases).

According to the present invention, methods for treating diseases or conditions associated with enhanced acquired immunity in subjects in need thereof, comprising administering a therapeutically effective amount of a STING ligand to the subjects are provided. According to the present invention, methods for inducing T cells to produce type I interferons in subjects in need thereof, comprising administering an effective amount of a STING ligand to the subjects are also provided. According to the present invention, methods for inhibiting proliferation of acquired immune lymphocytes in subjects in need thereof, comprising administering an effective amount of a STING ligand to the subjects are provided.

According to the present invention, use of STING ligands in the manufacture of pharmaceutical compositions for use in treating diseases or conditions associated with enhanced acquired immunity is provided. According to the present invention, use of STING ligands in the manufacture of pharmaceutical compositions for use in inducing T cells to produce type I interferons is also provided. According to the present invention, use of STING ligands in the manufacture of pharmaceutical compositions for use in inhibiting the proliferation of acquired immune lymphocytes is further provided. According to the present invention, use of STING ligands in treating diseases or conditions associated with enhanced acquired immunity is also provided.

The present invention is described referring to Examples, below. Examples described below may be one of the embodiments of the present invention.

### EXAMPLES

### Example 1: Acquired Immunity and STING Ligand

In this Example, the response of acquired immune lymphocytes to STING ligands was examined.

### (1) Cell Proliferation Inhibiting Effect

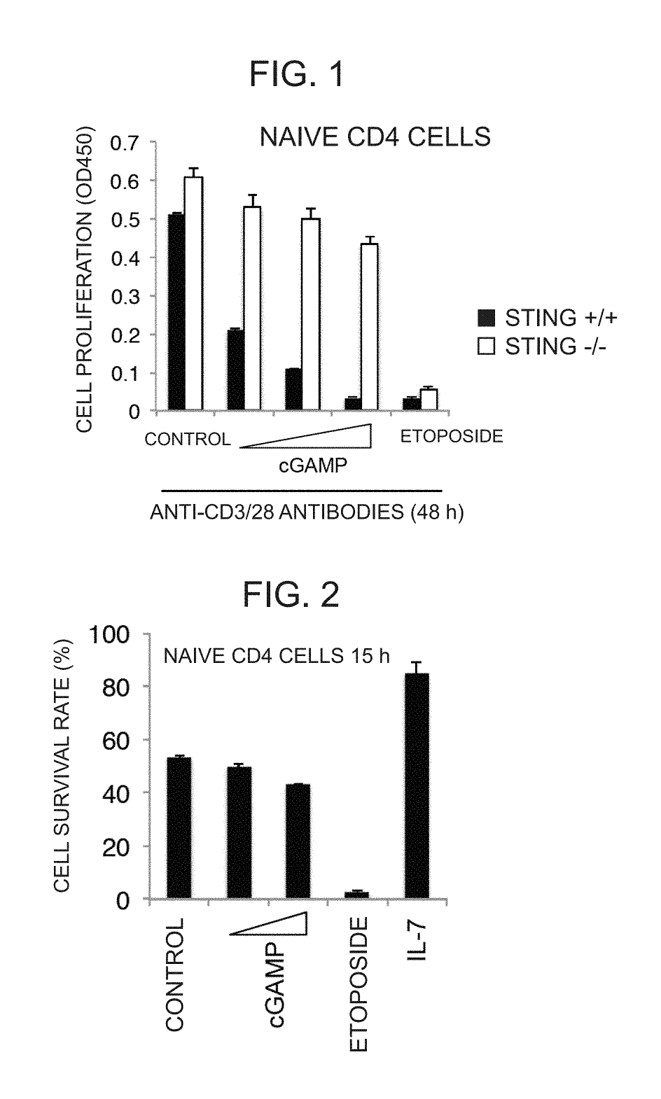

Wild type mice and STING-deficient mice were used. As acquired immune lymphocytes, T cells (in particular naïve CD4SP T cells) were used. Naïve CD4SP T cells were obtained as a CD4⁺ CD25⁻ CD62L⁺ fraction from murine spleen by sorting using FACSAria. As a STING ligand, cyclic [G(2′,5′)pA(3′,5′)p] (hereinafter, simply referred to as “cGAMP”) was used. cGAMP (manufactured by Invitrogen, Cat. No.: tlrl-nacga23-1) was used at a working concentration of 3 μg/mL, 10 μg/mL, or 30 μg/mL. The obtained T cells were stimulated with an anti-CD3 antibody (10 μg/mL) and an anti-CD28 antibody (10 μg/mL) in the presence or absence of cGAMP. As a control, etoposide (manufactured by Calbiochem, Cat. No.: 341205-25MGCN) was used at a concentration of 10 μM. The cells were treated for 48 hours and then the cell proliferation was measured by the optical density (OD 450 nm). The result was as illustrated in FIG. 1.

As illustrated in FIG. 1, the number of naïve CD4SP T cells decreased in a concentration-dependent manner in the cGAMP treatment group of wild type mice (STING+/+). In the STING-deficient mice (STING−/−), the effect of cGAMP on the number of naïve CD4SP T cells was limited. This revealed that cGAMP, a STING ligand, exerts its cell proliferation inhibiting effect on acquired immune lymphocytes through STING.

Moreover, the number of cells decreased independent of the STING genotype in the group treated with etoposide, an apoptosis inducer, and the treatment with cGAMP acted in a degree similar to etoposide.

### (2) Apoptosis Inducing Effect

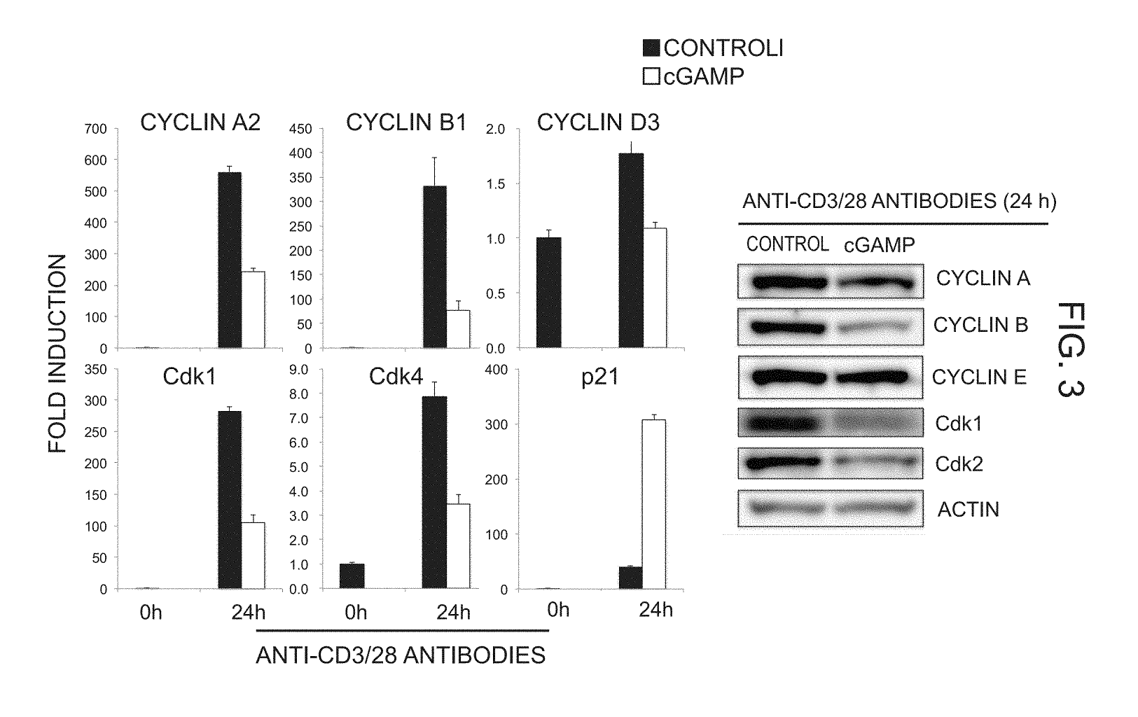

As described in (1) above, naïve CD4SP T cells were stimulated with 10 μg/mL or 30 μg/mL cGAMP, 10 μM etoposide, or 10 μg/mL IL-7 (manufactured by PeproTech, Inc., Cat. No.: 217-17) for 15 hours and then collected and the cell survival rate was measured. The result was as illustrated in FIG. 2.

As illustrated in FIG. 2, with etoposide a large quantity of cells died by apoptosis, but with cGAMP any prominent apoptosis-inducing effect was not found while the cell survival rate decreased to some extent in a concentration-dependent manner. This suggested that the effect of cGAMP on T cells is inhibition of cell proliferation rather than induction of cell death.

### (3) Change of Gene Expression

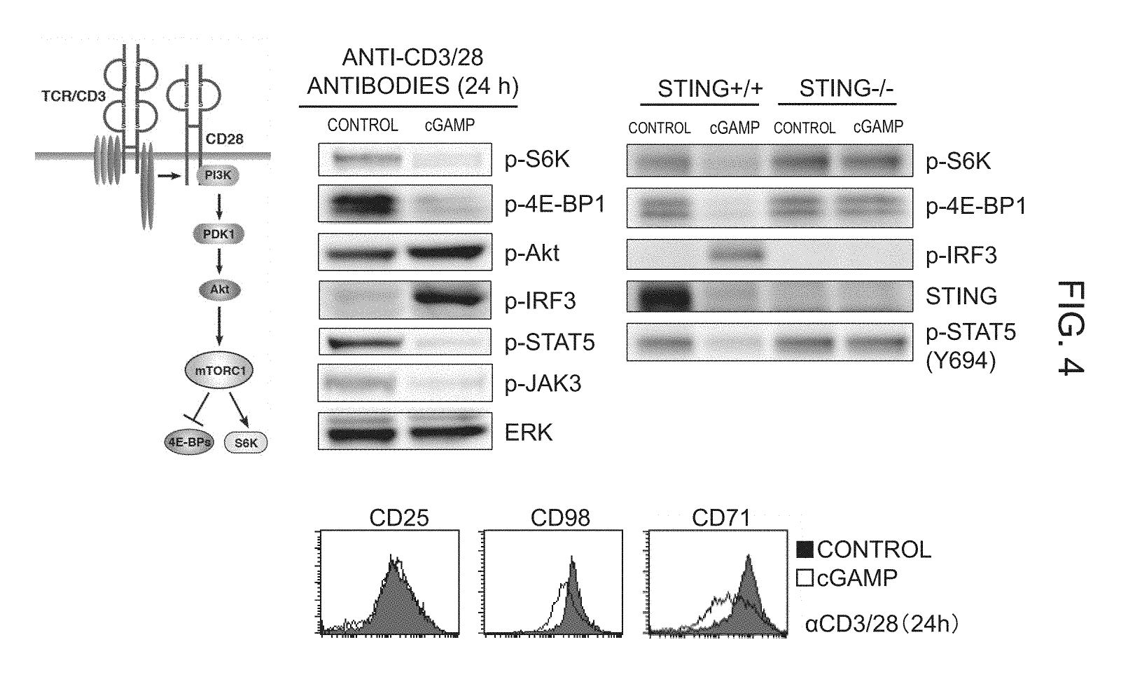

As described in (1) above, naïve CD4SP T cells were stimulated with anti-CD3/CD28 antibodies for 24 hours in the presence or absence of cGAMP. Subsequently, the gene expression of cell cycle-related factors was examined by quantitative PCR according to a conventional method. The result was as illustrated in FIG. 3.

As illustrated in FIG. 3, the expression of cyclin A2, cyclin B1, cyclin D3, Cdk1, and Cdk4 was inhibited by cGAMP. Meanwhile, the expression of p21 was increased by cGAMP. This revealed that cGAMP inhibits the expression of factors that positively control the cell cycle and increases the expression of factors that negatively control the cell cycle under T-cell stimulation. This suggested that the role of STING in T cells is to negatively control the cell cycle and to inhibit the cell proliferation.

(4) Relation with mTOR Complex 1 Signal

As a signal molecule that is involved in the expression of cell proliferation-related genes, the activation level of mTOR Complex 1 (mTORC1) signaling was analyzed. Specifically, as described in (1) above, naïve CD4SP T cells were stimulated with anti-CD3/CD28 antibodies for 24 hours in the presence or absence of cGAMP. Subsequently, the phosphorylated level of indicated factors was evaluated with antibodies specific to the phosphorylated form by western blotting. As primary antibodies, the antibodies set forth in the following were used. The following primary antibodies were manufactured by Cell Signaling Technology, Inc. and the catalog numbers are indicated in the parentheses.

Anti-phospho-S6K (#9205)

Anti-phospho-4E-BP1 (#9459)

Anti-phospho-Akt (#9271)

Anti-phospho-IRF3 (#4947)

Anti-phospho-STAT5 (#9351)

Anti-phospho-Jak3 (#5031)

Anti-phospho-STING (#13647)

As a second antibody, Goat anti-Rabbit IgG (H+L) Secondary Antibody, HRP from Thermo Fisher Scientific Inc. was used. Phosphorylated proteins were detected according to a manufacturer manual. The result was as illustrated in FIG. 4. As illustrated in FIG. 4, the levels of signal molecules (S6K and 4E-BP1), whose phosphorylated form is induced by T-cell receptor stimulation, decreased by cGAMP.

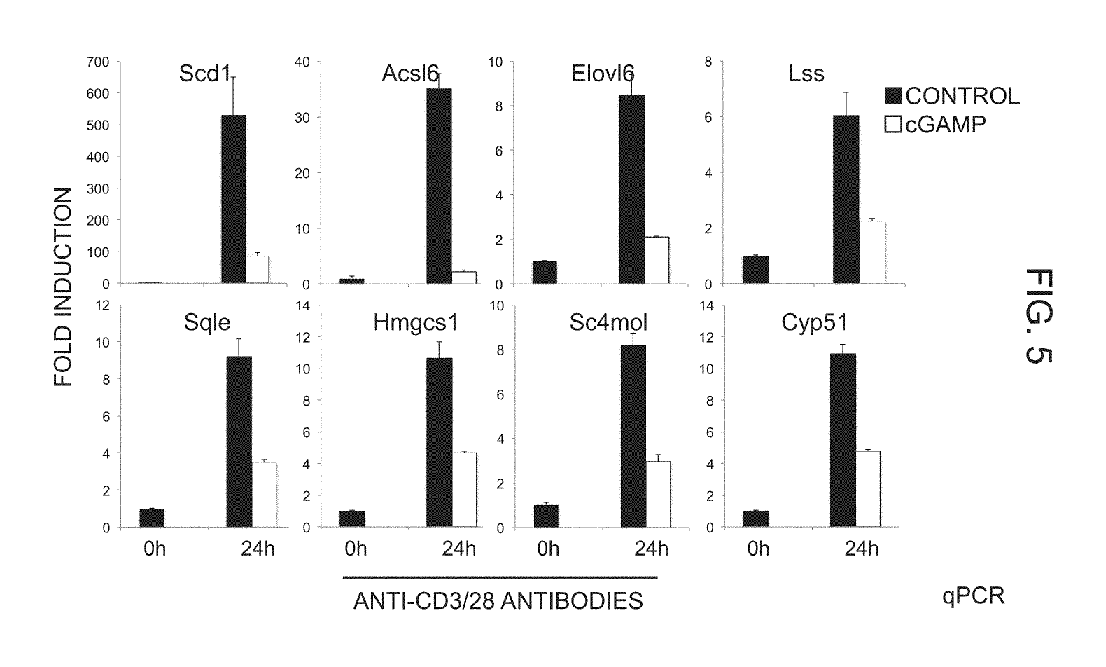

Moreover, the expression levels of lipid synthesis genes that are induced by the activation of mTORC1 were analyzed by quantitative PCR. The result was as illustrated in FIG. 5. As illustrated in FIG. 5, the expression levels of lipid synthesis-related genes decreased by cGAMP.

These results revealed that STING negatively controls the cell cycle and inhibits the cell proliferation of T cells by inhibiting the activation of mTORC1.

### Example 2: Production of Type I Interferon in Acquired Immune Lymphocytes

In T or B cells, no type I interferons are produced even when mTORC1 is activated. In this Example, it is illustrated that STING increases the type I interferon production.

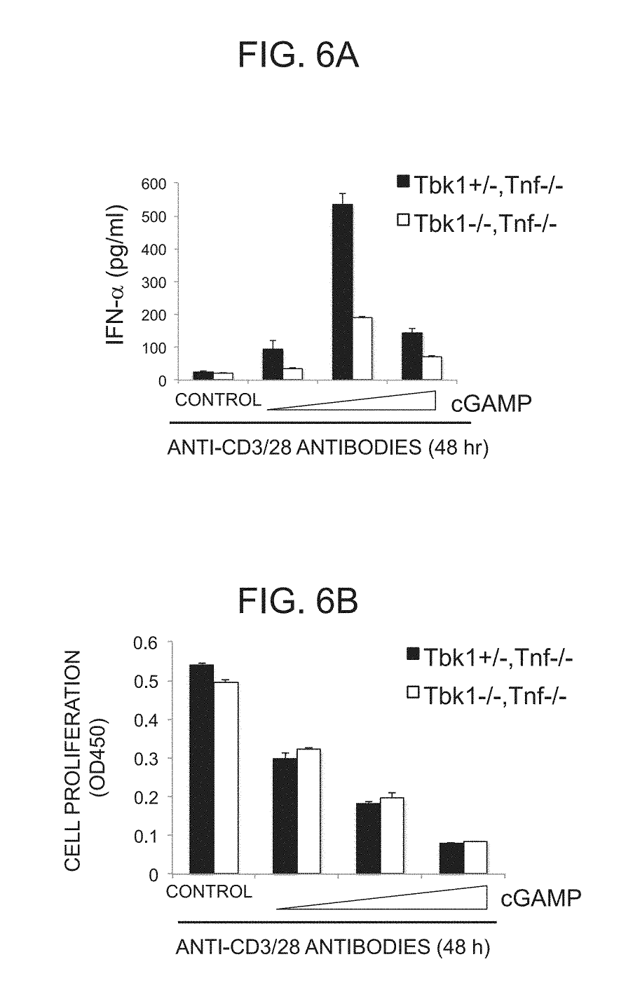

1×10⁵ naïve CD4SP T cells were stimulated for 48 hours on a plate on which an anti-CD3 antibody (10 μg/mL) and an anti-CD28 antibody (10 μg/mL) were immobilized. The cell supernatant was collected and the production of the type I interferon INF-α was examined by ELISA using an anti-INF-α antibody. In innate immune cells, type I interferons are produced by the activation of TBK1 and its downstream IRF3 and IRF7. Therefore, T cells from TBK1 heteromice (Tbk1+/−) and TBK1-deficient mice (Tbk1−/−) were used (see, J Exp Med, Vol. 199, 1641-1650, 2004). The result was as illustrated in FIG. 6A. As illustrated in FIG. 6A, prominent increase in INF-α production in T cells by cGAMP was unexpectedly found. Moreover, the increase in production of INF-α was decreased by TBK1 knockout. Therefore, it was revealed that the increase in INF-α production in T cells by cGAMP involves the activation of TBK1.

Moreover, the involvement of TBK1 in the function to inhibit cell proliferation by cGAMP was examined using TBK1 heterozygous mice and TBK1 knockout mice. The result was as illustrated in FIG. 6B. As illustrated in FIG. 6B, the knockout of Tbk1 had almost no effect on the cell proliferation.

These results revealed that TBK1 is partially involved in the effect of increasing INF-α production by cGAMP but not in the effect of inhibiting cellular proliferation by cGAMP.

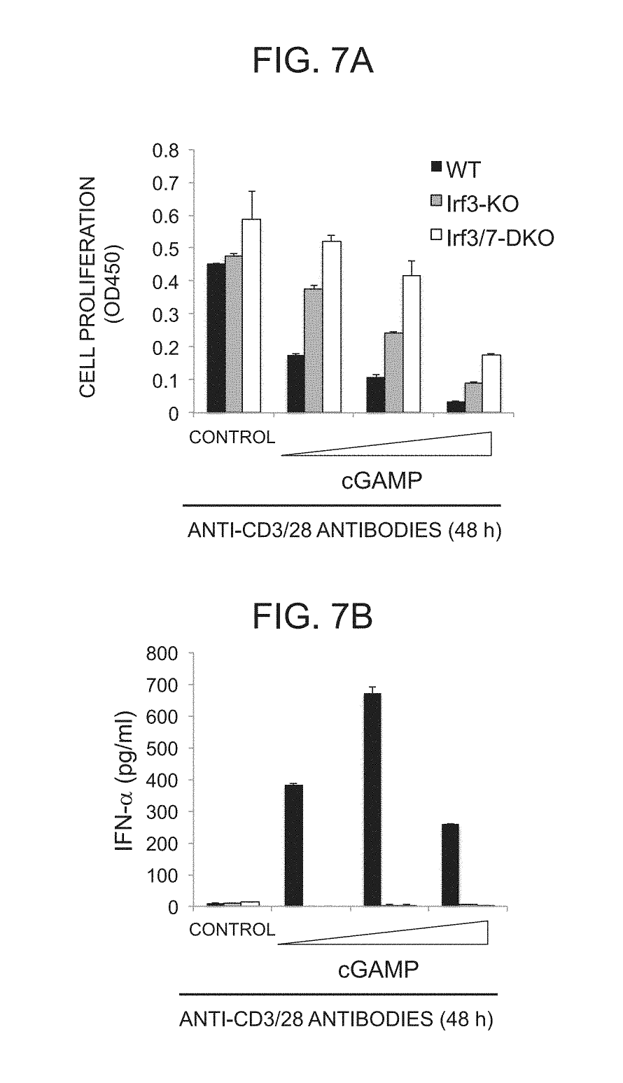

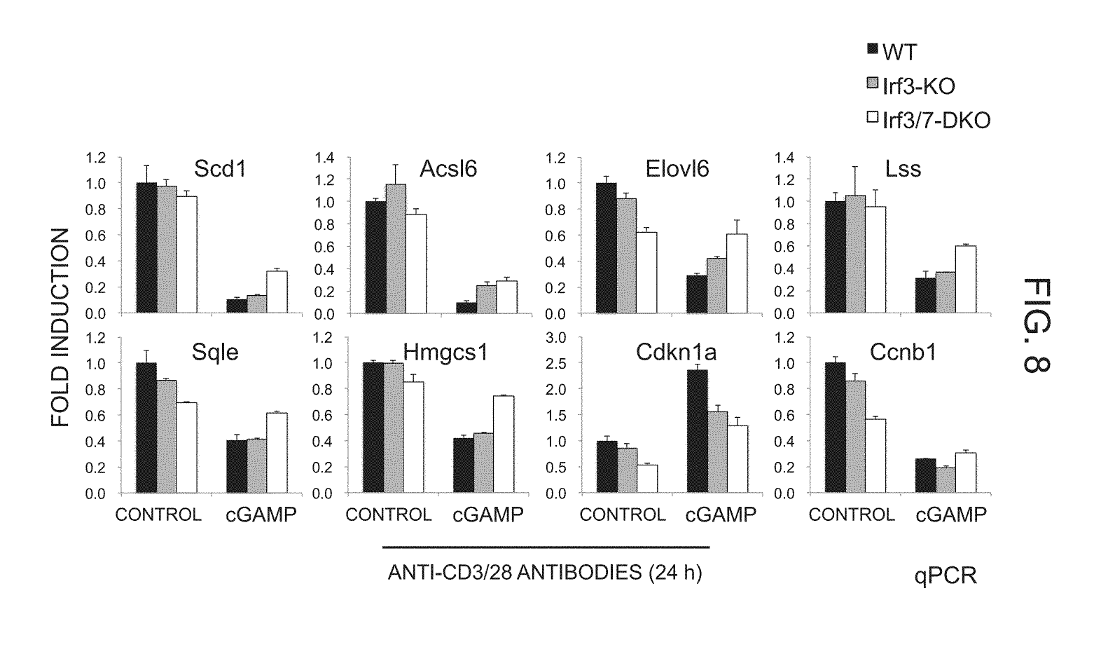

Next, involvement of IRF3 and IRF7 in the effect of inhibiting cellular proliferation and the effect of increasing production of INF-α through STING were examined. Specifically, T cells from IRF3-deficient mice or IRF3/IRF7 double deficient mice (see Immunity, Vol. 13, 539-548, 2000; Nature, Vol. 434, 772-777, 2005) were stimulated by anti-CD3/CD28 antibodies in the presence or absence of cGAMP. 48 hours later, the proliferation of the cells and the production of INF-α were examined as described above. The results were as illustrated in FIGS. 7A and 7B.

As illustrated in FIG. 7A, the effect of inhibiting cellular proliferation by cGAMP was largely decreased in the IRF3/IRF7 double deficient mice. Moreover, as illustrated in FIG. 7B, the effect of increasing production of INF-α by cGAMP almost completely disappeared in the IRF3/IRF7 double deficient mice. Moreover, as illustrated in FIG. 7A, the effect of inhibiting cellular proliferation by cGAMP partially decreased in the IRF3 deficient mouse. As illustrated in FIG. 7B, the effect of increasing production of INF-α by cGAMP almost completely disappeared in the IRF3 deficient mice.

These results revealed that the effect of inhibiting cell proliferation by STING involves inhibiting the activation of mTORC1 signals through IFR3 and IFR7.

The STING ligands inhibited the cell proliferation by the cell stimulation not only in innate immune cells but in acquired immune lymphocytes and exhibited the effect of increasing production of the type I interferon in T cells. In this Example, the effect of STING ligands to cell proliferation when the CD3/CD28 stimulation, which is stimulation similar to the T-cell receptor signal, was added to T cells was examined. In T cells, the STING ligand exhibited its physiological function through mTORC1 and its downstream IRF3 and IRF7. In B cells, similar mTORC1 signaling pathways exist and are activated under B-cell receptor stimulation. Therefore, it is suggested that STING ligands have similar functions also in B cells.

Thus, STING ligands will play the effect of inhibiting proliferation of acquired immune lymphocytes and the effect of increasing production of type I interferons. Therefore, STING ligands can be useful as an immunosuppressive agent for the acquired immune system and are considered to be useful in treating diseases or conditions by excessive enhancement of the acquired immune system. Since STING ligands have a strong effect of inhibiting proliferation of acquired immune lymphocytes, they can particularly be useful in prophylactic treatments of conditions before the onset or during the disease progression, not only after the disease onset.

1. A method for treating or for prophylactically treating a disease or condition associated with enhanced acquired immunity in a subject in need thereof, comprising administering an effective amount of a STING ligand to the subject.

2. The method according to claim 1, wherein the disease or condition associated with enhanced acquired immunity is an autoimmune disease or a graft versus host disease (GVHD).

3. The method according to claim 1, wherein the method is for prophylactically treating the disease or condition associated with enhanced acquired immunity in the subject in need thereof, and comprises administering the effective amount of the STING ligand to the subject before the onset of the disease or condition.

4. The method according to claim 1, wherein the STING ligand is cyclic GMP-AMP.

5. A method for inducing a T cell to produce a type I interferon in a subject in need thereof, comprising administering an effective amount of a STING ligand to the subject.

6. A method for inhibiting proliferation of acquired immune lymphocytes in a subject in need thereof, comprising administering an effective amount of a STING ligand to the subject.

7. The method according to claim 2, wherein the STING ligand is cyclic GMP-AMP.

8. The method according to claim 3, wherein the STING ligand is cyclic GMP-AMP.

9. The method according to claim 1, wherein the method is for treating the disease or condition associated with enhanced acquired immunity in the subject in need thereof, and comprises administering a therapeutically effective amount of the STING ligand to the subject.

10. The method according to claim 9, wherein the STING ligand is cyclic GMP-AMP.
